(19) United States
(12) Patent Application Publication
(10) Pub. No.: US 2008/0171981 A1
(43) Pub. Date: Jul. 17, 2008

(54) TAMPER EVIDENT CAP FOR A DRUG DELIVERY DEVICE

(75) Inventors: Abdul Wahid Khan, Lindenhurst, IL (US); Edward J. Lefebre, Port Orange, FL (US)

Correspondence Address: DARBY & DARBY P.C., P.O. BOX 770, Church Street Station, New York, NY, 10008-0770, US

(73) Assignee: ForHealth Technologies, Inc., Daytona Beach, FL (US)

(21) Appl. No.: 11/743,965

(22) Filed: May 3, 2007

Related U.S. Application Data
(60) Provisional application No. 60/884,448, filed on Jan. 11, 2007.

(51) Int. Cl. A61M 5/00

(52) U.S. Cl. 604/111

(57) ABSTRACT

According to one exemplary embodiment, a tamper evident cap for placement on a syringe includes a first section for placement about a luer tip of the syringe and a second section for placement above the luer tip when the cap is placed on the syringe. The second section has a slot formed therein that is defined by a ceiling and an opposing floor. The cap also includes a slider received within the slot and being slideable in the slot from at least an open position where the luer tip is accessible through the second section for filling the syringe and a locked position where the luer tip is covered by the slider and access to the luer tip is prevented.

### CROSS-REFERENCE TO RELATED APPLICATION

The present application claims the benefit of U.S. provisional patent application Ser. No. 60/884,448, filed Jan. 11, 2007, which is hereby incorporated by reference in its entirety.

### TECHNICAL FIELD

The present invention relates generally to medical and pharmaceutical equipment, and more particularly, to a tamper evident cap that is constructed to mate with a drug delivery device, such as syringe, to indicate whether the drug delivery device has been tampered with especially after the filling thereof.

### BACKGROUND

Drug delivery devices are commonly used to hold and deliver prescribed quantities of medication to a patient. Exemplary drug delivery devices include, but are not limited, to syringes, IV bags, and other containers that function to hold and deliver a prescribed and precise amount of medication to the patient.

Accordingly, disposable syringes are in widespread use for a number of different types of applications. For example, syringes are used not only to withdraw a fluid (e.g., blood) from a patient but also to administer a medication to a patient. In the latter, a cap or the like is removed from the syringe and a unit dose of the medication is carefully measured and then injected or otherwise disposed within the syringe.

As technology advances, more and more sophisticated, automated systems are being developed for preparing and delivering medications by integrating a number of different stations, with one or more specific tasks being performed at each station. For example, one type of exemplary automated system operates as a syringe filling apparatus that receives user inputted information, such as the type of medication, the volume of the medication and any mixing instructions, etc. The system then uses this inputted information to disperse the correct medication into the syringe up to the inputted volume.

In some instances, the medication that is to be delivered to the patient includes more than one pharmaceutical substance. For example, the medication can be a mixture of several components, such as several pharmaceutical substances.

By automating the medication preparation process, increased production and efficiency are achieved, as well as a reduction in the risk of contamination. This results in reduced production costs and also permits the system to operate over any time period of a given day with only limited operator intervention for manual inspection to ensure proper operation is being achieved. Such a system finds particular utility in settings, such as large hospitals, that require a large number of doses of medications to be prepared daily. Traditionally, these doses have been prepared manually in what is an exacting but tedious responsibility for a highly skilled staff. In order to be valuable, automated systems must maintain the exacting standards set by medical regulatory organizations, while at the same time simplifying the overall process and reducing the time necessary for preparing the medications.

Because syringes are used often as the carrier means for transporting and delivering the medication to the patient, it is advantageous for these automated systems to be tailored to accept syringes. However, the previous methods of dispersing the medication from the vial and into the syringe were very time consuming and labor intensive. More specifically, medications and the like are typically stored in a vial that is sealed with a safety cap or the like. In conventional medication preparation, a trained person retrieves the correct vial from a storage cabinet or the like, confirms the contents and then removes the safety cap manually. This is typically done by simply popping the safety cap off with one's hands. Once the safety cap is removed, the trained person inspects the integrity of the membrane and cleans the membrane. An instrument, e.g., a needle, is then used to pierce the membrane and withdraw the medication contained in the vial. The withdrawn medication is then placed into a syringe to permit subsequent administration of the medication from the syringe.

As is known, the safety of the patient is of utmost importance and therefore, the various medication processing and manufacturing equipment typically incorporate various safety features that indicate to a user (patient) whether the product may have been tampered with at an earlier time. For example, a container that houses solid medication, such as pills, tablets, or capsules, often includes a tamper proof label that extends and is sealed across the top opening of the container underneath the cap. Thus, when a consumer initially purchases the product and unscrews the cap, the tamper proof label should be fully intact and sealed across the opening of the container. If the label is not intact, the consumer should not use the medication contained therein and instead should report the incident and discard the bottle and its contents. Other types of tamper evident sealing are also known for indicating to the consumer or patient whether the product may have been tampered with and therefore, should not be used for the sake of safety.

While there are several products on the market that provide a tamper evident feature as part of a cap for a syringe, there are a number of deficiencies with these products. In particular, one type of product is in the form of a colored break-away sleeve that caps the syringe (e.g., an IV luer lock syringe). The colored sleeves can be provided and arranged in a tray prior to being mated with the syringes such that a person can cap the syringe directly by inserting the end of the syringe to be capped into one selected colored sleeve that is in the tray resulting in the colored cap being securely attached to this end. If the syringe is tampered with and the contents are accessed through the capped end, the colored sleeve will be broken, thereby providing an immediate visual indicator that the syringe has been tampered with. Another commercial product is similar in that the open end of the syringe is capped with a shell which is constructed and attached to the open end of the syringe such that tampering with the syringe results in the shell being broken away, thereby providing an immediate visual indicator that the syringe has been tampered with. Thus, in both of these products, a member, such as a perforated shell, is broken away in order to gain access to the interior of the barrel and therefore, it can immediately be apparent if the syringe has been tampered with since the shell will be broken away.

However, there are a number of disadvantages with these types of structures. For example, the placement of a shell on the open end of the barrel effectively seals off the syringe and therefore, the syringe must be filled prior to placing the shell on the syringe. Thus, the shell can not be added as part of the syringe manufacturing process since the shell can not be added to an empty syringe and the filling of a syringe, even in automated drug preparation systems, such as the one disclosed herein, occurs after the syringes are manufactured. The drug preparation process typically entails having a bulk supply (box) of the syringes and then filling the syringes with the prescribed quantities of proper medication. The disadvantage of using a shell as a means for providing a tamper evident feature is that the filling of the syringe cannot occur with the shell on the syringe and therefore, the positioning of the shell on the syringe must be incorporated into the post filling process and requires a separate station where a bulk supply of shells are located and when the filling is part of an automated process, additional automated equipment is needed for placing the shell on the syringe.

What is needed in the art and has heretofore not been available is a tamper evident cap that is particularly, suited for use with a syringe and can be incorporated into an automated medication preparation system that prepares and fills syringes with prescribed quantities of medication.

### SUMMARY

According to one exemplary embodiment, a tamper evident cap for placement on a syringe includes a first section for placement about a luer tip of the syringe and a second section for placement above the luer tip when the cap is placed on the syringe. The second section has a slot formed therein that is defined by a ceiling and an opposing floor.

The cap also includes a slider received within the slot and being slideable in the slot from at least an open position where the luer tip is accessible through the second section for filling the syringe and a locked position where the luer tip is covered by the slider and access to the luer tip is prevented.

In yet another embodiment, a tamper evident cap for placement on a syringe includes a hollow bottom base section for receiving a luer tip of the syringe such that the base section surrounds the luer tip of the syringe and an upper flange section that is integrally attached to the base section. The upper flange section has an opening formed therein that communicates with the base section and is located to provide an entrance into the luer tip when the cap is placed on the syringe. The upper flange section has a slot formed therein that is defined by a first surface and an opposing second surface.

The tamper evident cap includes a slider having a locking tab formed as a part thereof. The slider is received within the slot and is movable in the slot between a first position, a second position and a third position. In the first position, the slider is releasably retained in a closed position by reception of the locking tab in a first catch resulting in the luer tip being covered by the slider and not accessible through the flange section. In the second position, the luer tip is accessible through the opening of the upper flange section to permit filling of the syringe. In the third position, the slider is laterally locked in place within the slot by reception of the locking tab in a second catch resulting in the luer tip being covered by the slider and access to the luer tip is prevented. Movement of the slider in the slot subsequent to positioning the slider in the locked position causes the locking tab to break off of the slider, thereby indicating that the syringe has been tampered with subsequent to its filling.

### BRIEF DESCRIPTION OF THE DRAWING FIGURES

FIG. 1 is a side perspective view of a tamper evident cap according to one embodiment of the present invention;

FIG. 2A is a side cross-sectional view of a conventional syringe capped with the tamper evident cap with a slider thereof being shown in an initial first position (manufacturer position);

FIG. 2B is a side cross-sectional view of the tamper evident cap with the slider being shown in an open second position for filling the syringe;

FIG. 2C is a side cross-sectional view of the tamper evident cap with the slider being shown in a locked third position;

FIG. 3A is a side cross-sectional view of a conventional syringe capped with a tamper evident cap according to another embodiment with a slider thereof being shown in an initial first position (manufacturer position);

FIG. 3B is a side cross-sectional view of the tamper evident cap with the slider being shown in an open second position for filling the syringe;

FIG. 3C is a side cross-sectional view of the tamper evident cap with the slider being shown in a locked third position;

FIG. 4 is an exploded top and side perspective view of a tamper evident cap according to yet another embodiment with a cap top exploded from a cap body;

FIG. 5 is an exploded bottom and side perspective view of the cap of FIG. 4;

FIG. 6A is a top plan view of the cap top in an open (fill) position relative to the cap body;

FIG. 6B is a top plan view of the cap top in an intermediate position;

FIG. 6C is a top plan view of the cap top in a closed position;

FIG. 7A is a partial cross-sectional view of the cap top in the closed, unlocked position; and

FIG. 7B is a partial cross-sectional view of the cap top in a locked position.

### DETAILED DESCRIPTION OF PREFERRED EMBODIMENTS

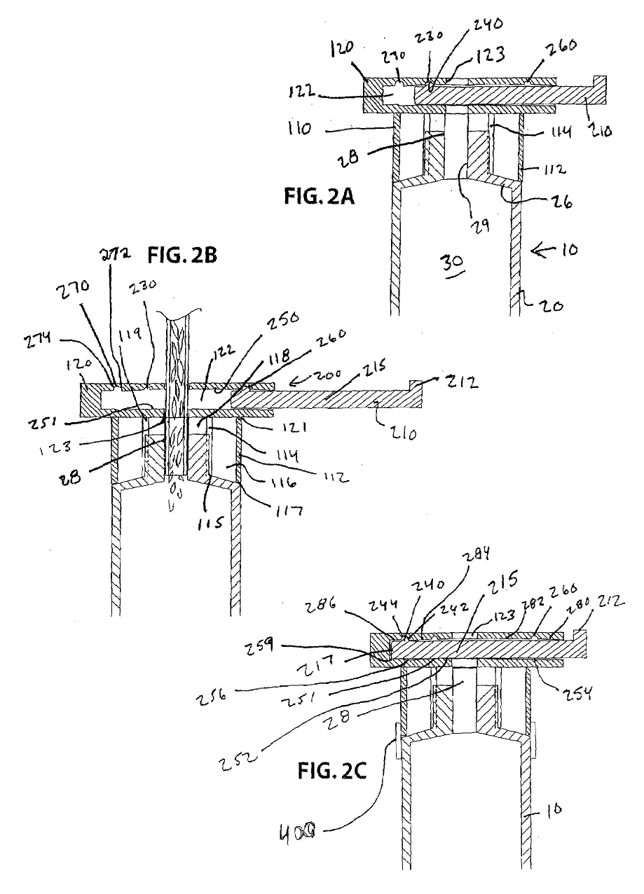

FIGS. 2A-2C illustrate an exemplary conventional syringe 10 that includes a barrel 20 having an elongated body that defines a chamber 30 that receives and holds a medication that is disposed at a later time. The barrel 20 has an open proximal end which can include a flange being formed thereat and it also includes an opposing distal end 26 that has a luer tip 28 that has a passageway 29 formed therethrough. One end of the passageway 29 opens into the chamber 30 to provide communication between the luer tip 28 and the chamber 30 and the opposing end of the passageway 29 is open to permit the medication to be dispensed through a cannula or conduit or other device (not shown) that is later coupled to the luer tip 28.

An outer surface of a base of the luer tip 28 can include features to permit fastening with a cap or other type of enclosing member. For example, the luer tip 28 can have threads that permit a tip cap to be securely and removably coupled to the luer tip 28 or the tip cap can be slid over the luer tip 28 and frictionally held thereon. The tip cap is constructed so that it closes off the passageway 29 to permit the syringe 10 to be stored and/or transported with a predetermined amount of medication disposed within the chamber 30. As previously mentioned, the term “medication” refers to a medicinal preparation for administration to a patient and most often, the medication is contained within the chamber 30 in a liquid state even though the medication initially may have been in a solid state, which was processed into a liquid state.

The syringe 10 further includes a plunger (not shown) that is removably and adjustably disposed within the barrel 20. A distal end of the plunger terminates in a stopper or the like that seals against the inner surface of the barrel 20 within the chamber 30. The plunger can draw a fluid (e.g., air or a liquid) into the chamber 30 by withdrawing the plunger from an initial position where the stopper is near or at the luer tip 28 to a position where the stopper is near the proximal end of the barrel 20. Conversely, the plunger can be used to expel or dispense medication by first withdrawing the plunger to a predetermined location, filling the chamber 30 with medication and then applying force against the flange so as to move the plunger forward within the chamber 30, resulting in a decrease in the volume of the chamber 30 and therefore causing the medication to be forced into and out of the luer tip 28.

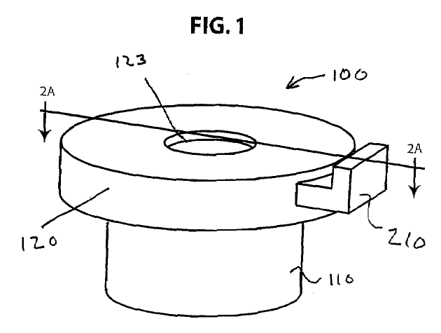

In accordance with one embodiment of the present invention and as illustrated in FIGS. 1 and 2A-2C, a tamper evident cap 100 is provided and is intended for use with the syringe 10. As described below, the tamper evident cap 100 is intended for placement on the syringe 10 at the point of manufacture and prior to filling the syringe 10 with a prescribed quantity of medication. The tamper evident cap 100 can be thought of as a luer slip fit cap 100 that is intended to mate with the luer tip 28 of the syringe 10 in a sliding, frictional fit manner such that the tamper evident cap 100 is slid over a base structure of the luer tip 28 to secure the cap 100 to the syringe 10.

The tamper evident cap 100 includes a base section 110 that is substantially hollow and a flange section 120 that is integrally attached to the base section 110. The base section 110 represents the bottom section of the syringe 10 and the flange section 120 represents the top section of the syringe 10.

The base section 110 is defined by a pair of concentric walls and more specifically, the base section 110 includes an outer annular wall 112 and an inner annular wall 114, with an annular space 116 formed therebetween. The inner wall 112 is a hollow member with a circular shaped space 118 being formed and defined by the inner wall 112. The height of the inner wall 114 is less than the height of the outer wall 112 since a bottom edge 117 of the outer wall 112 abuts and seats against the end of the barrel 30 of the syringe 10.

The inner wall 114 is constructed so that the space 118 is sized to receive the luer tip 28 to establish the frictional fit therebetween resulting in the cap 100 being held on the syringe 10. To couple the cap 100 to the syringe 10, the luer tip 28 is received into the space 118 and an inner surface of the inner wall 114 slidingly engages an outer surface of the base of the luer tip 28. The heights of the inner wall 114 and the outer wall 112 are such that when the cap 100 is placed on the luer tip 28 and the bottom edge 117 of the outer wall 112 seats against the barrel 30 of the syringe 10, a bottom edge 115 of the inner wall 114 seats against a point where the luer tip 28 joins the barrel 30 of the syringe 10.

The flange section 120 is joined to upper edges 119, 121 of the inner and outer walls 114, 112, respectively. The flange section 120 can come in any number of different shapes; however, the illustrated flange section 120 has a round, circular shape similar to the shape of the barrel 30 of the syringe 10. A diameter of the flange section 120 is greater than a diameter of the outer wall 112 and therefore, the flange section 120 extends beyond the upper edge 121 of the outer wall 112.

The flange section 120 is also constructed so that it houses a tamper evident feature 200 that permits an individual to immediately detect whether the syringe 10 has been tampered with after it has been filled with the prescribed medication. More specifically, the tamper evident feature 200 includes a slider 210 that is operably and slideable contained within a slot or space 122 formed in the flange section 120. As shown in FIGS. 2A-2C, the slider 210 includes a handle portion 212 to permit it to be easily grasped and manipulated within the slot 122 as described below. The flange section 120 is constructed so that it includes a central opening 123 formed therein which is aligned with and in communication with the space 118 that is defined by the inner wall 114. Since the space 118 receives the luer tip 28, the central opening 123 overlies the open luer tip 28 and therefore, a member that is received into the central opening 123 can travel into the open luer tip 28 and into the interior of the barrel 30. As a result of the foregoing, the location, shape and size of the central opening 123 is selected in view of the location, shape and size of the luer tip 28 since these two features must be in communication and aligned with one another. In this manner, the syringe 10 is filled with the prescribed quantity of medication. For example, a cannula can be inserted into the luer tip 28 for delivering medication to the syringe 10.

Optionally, the flange section 120 can have a window formed in the flange section 120 along an outer surface of a side wall thereof to permit viewing of the slider 210.

An opening of the slot 122 is formed in the outer surface of the side wall to permit reception of the slider 210 and to permit the sliding movement of the slider 210 within the slot 122. The handle portion 212 has a height that is greater than a height of the slot 122 and therefore, the handle portion 212 is not capable of being inserted into the slot 122.

In addition to the handle portion 212, the slider 210 has a body 215 that has a complementary shape to the flange section 120, as well as being of sufficient shape and size that covers the central opening 123 formed in the flange section 120 and covers the open luer tip 28 when the slider 210 is in a closed position. The body 215 can have a circular shape, a square shape, rectangular shape, etc.

The slider 210 is positioned in and slides within a plane that is at least substantially perpendicular to a longitudinal axis of the syringe that extends a length of the syringe through the luer tip 28 and the barrel 30. Preferably, as described below in greater detail, the slider 210 can be placed in a number of different positions including one or more closed positions where the open luer tip 28 is covered by the slider body 215 and an open position where the open luer tip 28 is open and accessible due to the slider body 215 being displaced from the open luer tip 28.

In accordance with one exemplary embodiment, the tamper evident feature 200 includes a locking feature that permits the slider 210 to be locked in a desired position relative to the flange section 120. In particular, the flange section 120 has a first recess, indentation or catch 230 that receives a protrusion or tab 240 that is formed as part of the body 215 of the slider 210 for positioning the slider 210 in a first position (initial position) in which the open luer tip 28 is covered, thereby ensuring the sterility of the syringe and any contents that may be present.

The first position can also be thought of as a manufacturing position in which the slider 210 is initially set during production of the syringe 10 and the mating or coupling of the cap 100 to the syringe 10. According to at least one embodiment, at the time that the slider 210 is in the first position and the cap 100 is mated to the syringe 10, the syringe 10 is empty.

The first position is also a position in which the slider 210 is capable of being freely moved to another position. In other words, when the slider 210 is placed in the first position by positioning the tab 240 into the first catch 230, the slider 210 can not freely move due to the mating of the tab 240 and the catch 230; however, if either a sufficient pull or push type force is applied to the slider 210, the tab 240 can be dislodged from the catch 230 and free to travel to another position without jeopardizing the structure and integrity of the tab 240.

As illustrated in FIGS. 2A-2C, the first catch 230 is formed along a ceiling portion 250 of the flange section 120 and in the illustrated embodiment, the first catch 230 has a generally hemi-spherical shape. The tab 240 has an arcuate, convex shape that departs from a true hemi-spherical shape in that that the height of a first end 242 of the tab 240 is less than a height of a second end 244 of the tab 240. The ceiling portion 250 partially defines the slot 122.

As a result, the radius of curvature of the first catch 230 is different from the radius of curvature of the tab 240 and therefore an exact mating between the two is not possible. In addition, the relative positioning and height of the first catch 230 relative to the tab 240 allows only a top portion of the tab 240 to enter the first catch 230. In this manner and once the tab 240 is received into the first catch 230, the slider 210 is locked in the first position in that there is sufficient resistance between the tab 240 and the first catch 230, that the slider 210 can not freely move within the slot 122. As mentioned below, in this first position, the slider body 215 is disposed over the entire open area of the luer tip 28, thereby closing off the luer tip 28. In the first position, a distal end 217 of the slider body 215 furthest from the handle portion 212 extends over and is sufficiently spaced from a peripheral edge of the luer tip 28 such that the luer tip 28 is completely closed off and covered. In this first position, there is some free space between the distal end 217 and the end of the slot 122 that receives the slider body 215 to permit the slider 210 to be further pushed into the flange section 120 in order to position the slider 210 in another position.

More specifically, the flange section 120 also includes a second recess, indentation or catch 260 that is formed along the ceiling portion 250. The second catch 260 receives the tab 240 for positioning the slider 210 in a second position in which the luer tip 28 is open and is accessible through the central opening 123 of the flange section 120.

The second catch 260 is similar in shape and size to the first catch 230 in that it has a generally hemi-spherical shape that is not identical to the shape of the tab 240. As with the first catch 230, the second catch 260 is positioned relative to the slider 210 such that when the tab 240 and second catch 260 are in registration with one another, only a portion of the tab 240 enters the second catch 260. However, the degree of mating between the tab 240 and the second catch 260 is sufficient to at least temporarily retain the slider 210 in the second position.

The second catch 260 is positioned in a location along the ceiling portion 250 such that the distal end 217 clears the hollow luer tip 28 and also clears the base of the luer tip 28 to which the inner wall 112 is engaged with for coupling the cap 100 to the syringe 10.

The generally hemi-spherical shape of the first and third catches 230, 270 and in particular, the smoothly curved inner surfaces thereof, permit the tab 240 to more easily slide into and out of engagement with the respective first and third catches 230, 270.

The flange section 120 further includes a third recess, indentation or catch 270 that is formed along the ceiling portion 250 such that the first catch 230 is located between the second and third catches 260, 270. The catches 230, 260, 270 are formed such that when the tab 240 is in the second catch 260, the length of the slider body 215 that lies outside of the flange section 120 is greater than when tab 240 is in one of the other catches 230, 270. When the tab 240 is disposed in the third catch 270, the length of the slider body 215 that lies outside the flange section 120 is at a minimum and thus, when the tab 240 is disposed in the first catch 230, the length of the slider body 215 lying outside the flange section 120 is in an intermediate range between the maximum and minimum.

The third catch 270 has a different shape and size compared to the first and second catches 230, 260 which may be of the same construction. In particular, the third catch 270 has a size and shape that are identical or closely similar to the size and shape of the tab 240 so that once the tab 240 is in registration with the third catch 270, the two mate together in a manner so that the amount of space (tolerance) between the tab 240 and the wall defining the third catch 270 is kept to a minimum. The third catch 270 thus has a first section 272 having a first recess depth and a second section 274 that has a second recess depth that is greater than the first depth. As a result, the radius of curvature of the third catch 270 is identical or substantially similar to the radius of curvature of the tab 240. The first section 272 is closer to the first catch 230, while the second section 272 is further therefrom.

Unlike, the generally hemi-spherical shape of the first and second catches 230, 270, the shape of the third catch 270 is such that the inner surface defining the third catch 270 is not as smoothly sloped and curved as the first and second catches 230, 270 (not as rounded) and therefore, once the tab 240 (which has similar rough edges) is received in the third catch 270, it is difficult to dislodge the tab 240 therefrom since the lack of smooth rounded surfaces prevents a smooth sliding action of the tab 240 over the inner surface.

When the slider 210 is in the third position (the tab 240 is disposed in the third catch 270), the luer tip 28 is completely closed off and covered by the slider body 215, thereby keeping the sterility of the product. It will be appreciated that this third position is one in which the slider 210 is locked in place after filling the syringe 10 with the product and unlike, when the slider 210 is in the first and second positions, the slider 210 can not freely move and easily be displaced from the third position due to the complementary shapes of the third catch 270 and the tab 240.

The slider 210 and cap 100 are constructed so that if the slider 210 is pulled back after the slider 210 has been placed in the locked third position, the tab 240 will break off of the slider body 215 showing that the syringe cap 100 has been tampered with. The tab 240 breaks off due to the irregular, rough shaped edges of the third catch 270 since the applied pulling force of the slider 210 is greater than a maximum force that the tab 240 can withstand without the tab 240 breaking off of the slider body 215. Once the tab 240 is broken off, a person can easily pull the slider 210 and in fact can completely remove the slider 210 from the flange section 120, thereby indicating that the syringe was tampered with after being filled since the slider 210 has been displaced from the locked third position.

This advantageously provides a tamper evident feature that not only is easy to see and determine if the syringe 10 has been tampered but also permits the syringe 10 to be filled with medication after the cap 100 has been placed on and mated to the syringe 10. As mentioned herein, this is not the case with other traditional tamper evident cap systems where the caps have to be placed on after the medication has been delivered to the syringe 10.

The ceiling portion 250 is not planar in that it contains or more beveled or angled portions. In particular, the illustrated ceiling portion 250 contains 4 distinct sections, namely, a first section 280 that is formed between the handle portion 212 and the second catch 260, a second section 282 that is formed between the first and second catches 230, 260, a third section 284 that is formed between the first and third catches 230, 270 and a fourth section 286 that is formed between the third catch 270 and an end of the slot 122 formed in the flange section 120.

In all of the sections 280, 282, 284, 286, the distance between the ceiling portion 250 and an opposite floor portion 251, both of which define the slot 122, is at least equal to or slightly greater than a maximum height of the slider body 215 and the tab 240. This permits reception of the slider 210 into the slot 122 and allows the slider 210 to slidingly move within the slot 122.

The first section 280 is constructed so that it defines an upwardly sloped ramp that leads to the second catch 260. At the peripheral edge of the flange section 120 which also defines one end of the first section 280, the height of the slot 122, as defined by the distance between the ceiling portion 250 and the opposite floor portion 251, is only slightly greater than the thickness of the slider body 215.

The second section 282 is a substantially planar portion in that the second section 282 is formed in a plane that is parallel to a plane that contains the floor portion 251 along the length of the second section 282. The distance between the ceiling portion 250 and the floor portion 251 is selected so that the tab 240 can slidingly travel along the ceiling portion 250 within the second section 282 to permit the slider 210 to move from the closed first position (first catch 230) to an open position (second catch 260) where the medication can be delivered to the luer tip 28.

In another aspect of one embodiment of the present invention, the floor portion 251 of the flange section 120 is not a completely planar surface although it does contain several planar sections. In particular, the illustrated floor portion 251 has two sections that depart from a planar section 252 of the floor portion 251. The floor portion 251 has a ramped section or surface 254 that is opposite the first section 280 of the ceiling portion and is at the opening into the slot 122 of the flange section 120. The ramped surface 254 leads up to the planar section 252. The floor portion 251 also has an indented or recessed portion 256 that is generally opposite the third section 284 in that the recessed portion 256 is located in a region between the first and third catches 230, 270. The recessed portion 256 represents a slight dip along the floor portion 251 where the height of the slot 122 is greater than surrounding portions due to the increased distance between the surface of the recessed portion 256 and the ceiling portion 250.

The recessed portion 256 is positioned along the floor portion 251 so that when the tab 240 is received in the first catch 230, the distal end 217 of the slider body 215 flexes into the recessed portion 256 to permit the slider body 215 to be accommodated in the slot 122 as described above. The smooth slope of the recessed portion 256 permits the distal end 217 to smoothly both into and out of the recessed portion 256 as the slider 210 is moved within the slot 122 of the flange section 120. When the tab 240 is disposed within the third catch 270, the distal end 217 of the slider body 215 is positioned along a section 259 of the floor portion 251 that is opposite the fourth section 286 of the ceiling portion 250 and is planar in that it is parallel to the fourth section 286 and is within the same plane as the planar section 252. In this position, there will be a slight open space below the slider body 215 in the region of the recessed portion 256.

The constructions of the floor portion 251 and the ceiling portion 250 permit the slider 210 to move in the intended, desired manner and more specifically, the slider 210 can move from the manufacturer's position where the luer tip 28 is closed to an open position where medication can be delivered to the syringe and then to a locked position where the slider 210 is locked and covers and closes off the luer tip 28.

Preferably, the cap 100 is secured to the syringe 10 by placing the cap 100 over the luer tip 28, as described above, such that the inner wall 112 of the cap 100 frictionally engages the base of the luer tip 28 and then a securing member 400 can be applied for securing the cap 100 on the syringe 10. In the illustrated embodiment, the securing member 400 is in the form of a flexible member 400 that is applied such that at least a portion of the securing member 400 is in contact with the syringe 10 (e.g., outer surface of the barrel 30) and another portion is in contact with the base section 110 of the cap 100. According to one exemplary embodiment, the flexible member 400 is in the form of a material that is provided in strips and can be wounds around the syringe 10 and the cap 100 for securely attaching the cap 100 to the syringe 10. The flexible member 400 can thus be in the form of a shrink wrap material that is provided in strip form and then is operated on to “shrink wrap” the strip to the cap 100 and syringe 10 resulting in the cap 100 being securely attached to the syringe 10 as shown in FIG. 2C. However, it will be appreciated that other types of material can be used as the flexible member 400.

The flexible member 400 is preferably adhered to the syringe 10 during the manufacturing process and not only does it serve to assist in attaching the cap 100 to the syringe 10 but it also provides a tamper evident feature in that it is to break if the cap 100 is attempted to be removed from the syringe 10 after manufacturing of the syringe 10, this indicates that the syringe 10 has been tampered with.

The distal end 217 of the slider body 215 can have rounded edges that assist in the slider 210 moving within the slot 122 and in particular, assist in the slider body 215 sliding across the floor portion 251 and in particular, in entering and exiting the recessed floor portion 256.

In yet another aspect, the syringe 10, with cap 100 attached thereto, can be incorporated into an automated drug preparation system, such as the one disclosed in commonly assigned U.S. patent application Ser. No. 11/434,850, which is hereby incorporated by reference in its entirety. In this manner, the syringes 10 capped with the caps 100 are introduced into the automated drug preparation system at a syringe loading station and then the automated components of the system operate on the syringe as described in the '850 application. For example, the slider 210 of the cap 100 can be manipulated so as to open the luer tip 28 (e.g., by pulling the slider 210 from the initial manufacturer's position) and then the prescribed quantity of medication is delivered to the syringe 10 through the luer tip 28. Alternatively and as described in the '850 application, the syringe 10 with cap 100 positioned thereon (but not secured with the flexible member) can be loaded into the automated drug preparation system and then a decapper apparatus removes the cap 100 prior to delivering the medication to the syringe and then subsequently, the cap 100 can be placed back on and the flexible member (shrink wrap strip) can be applied.

It will be appreciated that the syringes 10 can be loaded into the automated components in either a capped form where the cap 100 is on the luer tip 28 of the syringe 10 or in an uncapped form where the cap 100 is displaced from and not a part of the loaded syringe 10. In this situation, the uncapped syringes 10 are loaded onto the automated drug preparation system and then the syringes 10 can be filled prior to placement of and securing the cap 100 to the syringe 10 or the cap 100 can first be positioned on and secured to the syringe 10 prior to the filling thereof.

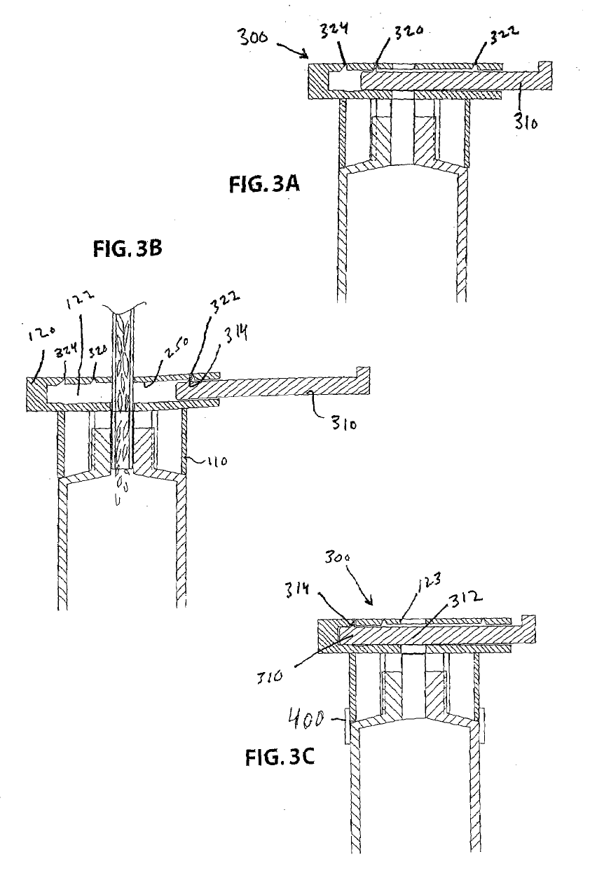

FIGS. 3A-3C illustrate another embodiment in which a cap 300 is provided. The cap 300 is similar to cap 100 with the difference being in the shape of the slider and the shape of the three catches that define the three positions of the slider. More specifically, in this embodiment, a slider 310 is provided that includes a body 312 having a locking tab 314 formed at a distal end of the slider 310. The locking tab 314 has a triangular shape and can be oriented so that a hypotenuse thereof faces towards the distal end of the slider body. In particular, the locking tab 314 can have the shape of a right angle triangle.

The cap 300 is formed so that the three catches formed along the ceiling portion 250 that defines the slot 122 are shaped and positioned to either releasably retain the tab 314 as in the case of first and second catches 320, 322, respectively. A third catch 324 is in the form of a triangular shaped recess (e.g., a right angle triangle) that is complementary and substantially the same shape as the tab 314, while the catches 320, 322 are not in the form of right angle triangles. For example, a rearward edge of the catch 320, 322 that faces the opening of the slot 122 has an angle other than 90 degrees when measured relative to the planar ceiling portion 250.

In the same manner as the previous embodiment, the locking tab 314 fits with less tolerance within the third catch 324 and therefore, once the locking tab 314 is received in the third catch 324, it is locked in place and cannot be displaced therefrom absent breaking off the tab 314 from the slider body. In contrast, when the tab 314 is disposed in either of the catches 320, 322, the slider 310 is retained in a respective position; however, it can be dislodged therefrom to laterally move the slider 310 without breaking off the tab 314.

It will also be appreciated that the tab formed on the slider body can also have more of a conical shape or some other shape, both regular and irregular shaped, so long as the shape of the tab is not identical or close to the shape of the first and second catches, but is similar to identical to the shape of the third catch (locked position).

The tamper evident cap 100 thus provides a marked improvement over conventional tamper evident caps due to it providing the following advantages and including the following features: (1) the cap 100 can be placed on the syringe 10 during the manufacturing phase; (2) the cap 100 is secure and in-place during sterilization, shipping and over the product shelf life; (3) the cap 100 is in place after processing the syringe in an automated drug preparation system; and (4) the cap 100 remains securely attached to the syringe 10 during the drug shelf like and in a freezer at temperature of −20 degrees C. to −40 degrees C.

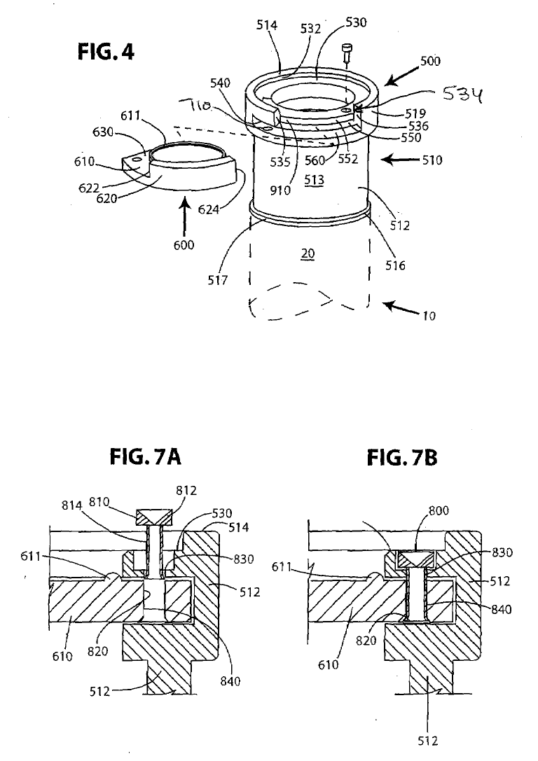

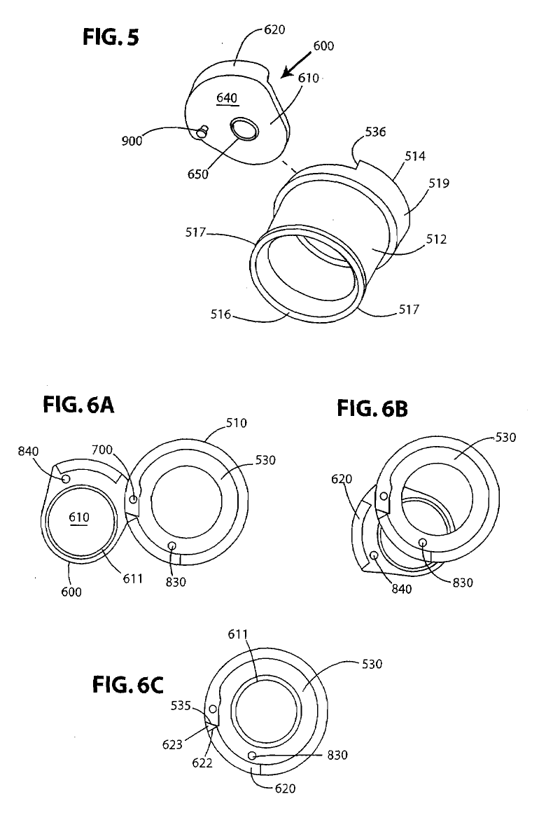

Now referring to FIGS. 4-7B in which a tamper evident cap assembly 500 according to yet another embodiment is illustrated. The cap assembly 500 includes a cap body 510 and a removeable cap top 600 that mates with the cap body 510 as described below. The cap body 510 is generally a hollow structure defined by a side wall 512 that is open at a first end (upper end) 514 and at a second end (lower end) 516. The shape and dimensions of the cap body 510 have to be complementary to the shape of the structure to which the cap body 510 is secured to and therefore, in the case of a syringe 10 and barrel 20 thereof, the cap body 510 has a cylindrical shape. Accordingly, the side wall 512 can be annular shaped.

At or near the open second end 516, an annular shaped lip 517 is formed along the outer surface of the side wall 512. Similarly, at or near the open first end 514, an annular shaped lip 519 is formed along the outer surface of the side wall 512. Preferably, the side wall 512 has a smooth surface in an area 513 between the first and second lips 517, 519. This area 513 is configured to receive the securing member, such as securing member 400 (FIG. 2C), which is used to secure the cap body 510 to the syringe barrel 20. In one embodiment, the securing member 400 is in the form of tamper evident securing tape to secure the cap body 510 to the syringe barrel 20.

The lips 517, 519 can form a right angle shoulder with the side wall 512 as illustrated. In the illustrated embodiment, the both lips 517, 519 are ring shaped structures that have diameters greater than a diameter of the cap body 510 in the area 513. In addition and as illustrated, the diameter of the lip 517 is less than the diameter of the lip 519.

Near the open first end 514, a ledge, platform or planar land 530 is formed. Since the cap body 510 has a cylindrical shape, the land 530 has an annular shape. The land 530 surrounds the opening that extends through the cap body 510. The land 530 is recessed relative to the first end 514 of the side wall 512 which is defined by an upper edge of the lip 519. A right angle shoulder 532 is formed between the land 530 and the side wall 512 portion that terminates in the first end 514.

In addition, the side wall 512 includes a slot 540 that is formed in the second lip 519 section. The slot 540 only partially extends around a circumference of the side wall 512. The slot 540 forms an entrance into the hollow interior of the cap body 510. The slot 540 is defined by an upper rail 550 that has an arcuate shape and is defined by the side wall 512 and a lower wall 560 that also has an arcuate shape and is defined by the side wall 512.

An upper surface 552 of the upper rail 550 defines a portion of the land 530. A portion 534 of the side wall 512 is removed above the upper rail 550 so as to define a break in the top part of the lip 519 and an opening into the interior of the side wall 512. Two edges 535, 536 define the portion 534 that is removed from the side wall 512 to define the upper rail 540. The edge 535 can be a beveled edge and the edge 536 is flat edge.

The cap body 510 can be formed of any number of different materials including different plastics. Likewise, the cap body 510 can be formed using any number of different processes, including a molding process, such as injection molding.

The cap top 600 is constructed so as to be removeably received within the slot 540 and is configured to be received into and to close off the portion 534. The cap top 600 has an irregular shape (e.g. wedge or pie shaped) and includes a base section 610 that includes a side wall 620 that extends partially around a portion of the perimeter of the base section 610. The base section 610 is defined by a first face or surface 630 and an opposing second face or surface 640. In the assembled position when the cap top 600 is inserted into the slot 540 and engages the cap body 510, the first face 630 is a top surface that faces the first end 514 of the cap body 510, while the second face 640 faces the second end 516 of the cap body 510.

The side wall 620 is a vertical peripheral wall segment that extends upwardly from the first face 630. A first edge (end) 622 of the side wall 620 is beveled, while a second edge (end) 624 is a flat edge or end. When the cap top 600 is inserted into the slot 540, the beveled edge 535 is proximate and spaced from the beveled edge 622 so as to define a gap or slot or wedge 623 (FIG. 6C) and the beveled edge 536 is adjacent the second edge 624 (no or little space existing therebetween).

The cap top 600 is dimensioned so that when it is received in the slot 540 and the side wall 620 mates with the side wall 512 so as to substantially complete the annular shape of the side wall 512, with the exception of the wedge 623, the base section 610 of the cap top 600 closes off the first end 514 of the cap body 510 and therefore prevents anyone from accessing the barrel tip of the syringe 10 that is capped by the cap assembly 500 of the present invention as described below. It will be appreciated that the side wall 620 is used to open and close the cap top 610 relative to the cap body 510.

Extending from the second face 640 is a pivot member 700 that permits the cap top 600 to pivot relative to the cap body 510. The pivot member 700 can be in the form of a protrusion or projection that extends outwardly from the second face 640. In the illustrated embodiment, the pivot member 700 is a circular shaped pivot pin extending outwardly from the second face 640. The pivot member 700 is configured to permit the cap top 600 to pivot between an open position and a closed position within the slot 640. In other words, when the cap top 600 is pivoted to the closed position, the cap top 600 completely closes off the slot 540, while when the cap top 600 is pivoted to the open position, the slot 540 is at least partially open.

Since the cap top 600 pivots relative to the cap body 510, an opening or bore 710 must be formed to receive the pivot pin 700. More specifically, the bore 710 is formed within the lower wall 560. The bore 710 and pin 700 are constructed to have complementary shapes so that once the pin 700 is received within the bore 710, the cap top 600 is engaged to the cap body 510 and can pivot relative thereto. In the illustrated embodiment, the bore 710 is a circular shaped bore.

As shown in FIG. 6A, in order to fill the syringe 10 with a liquid (e.g., medication), the cap top 600 is pivoted about the pin 700 so as to pivot the cap top 600 to an open position. In the open or fill position, the base section 610 of the cap top 600 does not completely occlude or close off the first end 514 of the body 510 but instead the barrel tip of the syringe 10 is accessible through the open first end 514 of the body 510. A liquid (medication) transfer device is mated with the barrel tip and can be used to deliver a prescribed amount of liquid into the syringe barrel.

The cap top 600 also preferably includes a location and sealing feature 650 that mates with and seals the luer tip 28 of the syringe 10. More specifically, the bottom surface or face 640 of the base section 610 includes a raised annular shaped protrusion or wall 650. The annular wall 650 is shaped and dimensioned so to frictionally receive and engage the luer tip 28 of the syringe 10 when the cap 500 is mated to the barrel 30 of the syringe 10. In other words, a friction fit is formed between the luer tip 28 and the annular wall 650. The height of the annular wall 650 is selected so that it does not interfere with the lower wall 560 of the cap body 510 when the cap top 600 is inserted into the slot 540 as when it is moved between the open fill position and the closed, locked position.

The cap top 600 has a locating and retention member 611 that is formed on the upper surface 630 of the base section 610 and is in the form of a projection or protrusion that extend outwardly from the upper surface 630. In the illustrated embodiment, the retention member 611 is in the form of an annular shaped lip that is located on the upper surface 630 so that when the cap top 600 closes, the lip 611 engages the opening defined by the inner edge of the annular shaped platform 530. More specifically, the lip 611 is received within the opening and frictionally engages the inner edge of the platform 530 so as to help retain the cap top 600 in the closed position prior to the cap top being locked as described below.

In addition and as best shown in FIGS. 7A and 7B, the cap assembly 500 includes a locking feature or mechanism 800 that selectively locks the cap top 610 relative to the cap body 510. The locking mechanism 800 includes a lock pin 810 that includes a head 812 and a stem 814 that extends therefrom. The head 812 can be a cylindrically shaped structure and the stem 814 can be a cylindrically shaped structure. The stem 814 can be at least partially hollow. At a free distal end of the stem 814, a lock flange 820 is formed around the outer surface of the stem 814. The flange 820 is in the form of an annular shaped lip that has a greater diameter than the diameter of the surrounding stem 814. The diameter of the head 812 is greater than the diameter of the stem 814. The lock pin 810 is formed of a material that has some resiliency.

The locking mechanism 800 also includes a first locking opening or bore 830 that extends through the upper rail 550 of the body 510. A second locking opening or bore 840 is formed in the base section 610 of the cap top 600. In one embodiment, the bore 840 is a through hole that extends completely through the base section 610 as shown in FIGS. 7A and 7B. Alternatively, the second locking opening or bore 840 does not pass completely through the base section 610 but instead it terminates near the bottom face 640 of the base section 610. Near a floor or closed end of the second locking bore 840 has an enlarged diameter section that is designed to receive the complementary shaped flange 820. The enlarged section is an annular shaped recess at the end of the bore 840.

When the cap top 610 is fully inserted into the slot 540 and closes off the first end 514 of the cap body 510, the first and second locking bores 830, 840 are axially aligned. When the lock pin 810 is inserted into the aligned bores 830, 840, the stem 814 travels within the bores 830, 840 and the flange 820 is slightly compressed. As soon as the flange 820 clears the bore 840 and is on the underside of the base section 610 (or is in registration with the enlarged section in the other embodiment), the flange 820 flexes outwardly resulting in a locking action between the lock pin 810 and the cap top 600. Since the head 812 of the lock pin 810 rests against the upper rail 550 of the cap body 510, the lock pin 810 serves to securely attach the cap top 600 to the cap body 510. In other words, the cap top 600 can no longer be freely pivoted open about pivot pin 700 so as to prevent access to the underlying syringe 10. In this manner, the cap 500 is a tamper evident cap in that if the cap top 600 is forcibly opened after the lock pin 810 is inserted and placed in the locked position, the consumer will notice that the lock pin 810 has been broken or is otherwise not in the locked position securing the cap top 610 to the body 510.

The cap assembly 500 can be placed on the syringe barrel by using either an automated apparatus or by placing them manually on the syringe barrel.

It is contemplated that in an initial shipping mode, the cap top 600 will be fully inserted into the slot 540; however, the lock pin 810 is only inserted through the first locking bore 830 that is formed through the upper rail 550 and is not inserted into the second locking bore 840. This orientation permits the cap top 600 to pivot to the open position (fill position) to permit filling of the syringe 10 as described above. This orientation can also be referred to as a “pin up” position. After opening the cap top 600 and filling the syringe with liquid (medication), the cap top 600 is closed and then the lock pin 810 is advanced into the second bore 840 to cause a frictional, locking fit between the stem flange 820 and the recess or groove 850.

It will be appreciated by persons skilled in the art that the present invention is not limited to the embodiments described thus far with reference to the accompanying drawings; rather the present invention is limited only by the following claims.

What is claimed is:

1. A tamper evident cap for placement on a syringe comprising:
a first section for placement about a luer tip of the syringe;
a second section for placement above the luer tip when the cap is placed on the syringe, the second section having a slot formed therein that is defined by a ceiling and an opposing floor; and
a slider received within the slot and being slideably movable in the slot from at least an open position where the luer tip of the syringe is accessible through the second section for filling the syringe and a locked position where the luer tip is covered by the slider and access to the luer tip is prevented, wherein movement of the slider from the locked position results in the slider being structurally modified to indicate the syringe has been tampered with.

2. The tamper evident cap of claim 1, wherein the first section is a base section that includes an inner annular wall that is constructed to be frictionally mounted to the luer tip which is received between the inner annular wall.

3. The tamper evident cap of claim 2, wherein the first section includes an outer annular wall that surrounds and is concentric with the inner annular wall.

4. The tamper evident cap of claim 3, wherein a height of the inner annular wall is less than a height of the outer annular wall.

5. The tamper evident cap of claim 1, wherein the first section is substantially hollow and the second section includes an opening that communicates with the second section.

6. The tamper evident cap of claim 1, wherein the slot has an opening that is accessible along an outer wall of the first section.

7. The tamper evident cap of claim 1, wherein the slider has a handle portion that has a height greater than a height of the slot so as to position the handle portion outside of the slot and the first section.

8. The tamper evident cap of claim 1, wherein the slider includes a first interlocking feature and the second section includes a plurality of second interlocking features for positioning the slider in one of the open and closed positions.

9. The tamper evident cap of claim 8, wherein the first interlocking feature comprises a tab formed along and protruding outwardly from a body of the slider and the second interlocking features include a first catch formed along the ceiling, the first catch receiving the tab to releasably retain the slider in a closed position in which the luer tip is covered and not accessible.

10. The tamper evident cap of claim 9, wherein a shape of the first catch is different from a shape of the tab and the tab is only at least partially received in the first catch to permit the slider to move laterally within the slot once a sufficient force is applied to the slider to cause the tab to become dislodged from the first catch without damaging the tab.

11. The tamper evident cap of claim 10, wherein the first catch has a substantially hemi-spherical shape and the tab has a convex surface with a varying radius of curvature.

12. The tamper evident cap of claim 9, wherein the second interlocking features include a second catch that interlockingly receives the tab to retain the slider in the locked position in which the luer tip is covered by the slider and is not accessible, the mating between the tab and the second catch being of a type that in order for the tab to be dislodged from the second catch, a pull force sufficient to break off the tab from the slider must be applied to the slider.

13. The tamper evident cap of claim 12, wherein the second catch has a shape that is substantially the same as a shape of the tab, the tab having a convex surface with a varying radius of curvature and the second cavity having a concave surface of varying radius of curvature.

14. The tamper evident cap of claim 12, wherein the second catch is located further from an opening of the slot in a side wall of the second section compared to the first catch.

15. The tamper evident cap of claim 12, wherein the first and second catches are recesses formed in the ceiling and a portion of the ceiling between the first and second catches is a beveled surface that declines in a direction from the first catch to the second catch.

16. The tamper evident cap of claim 15, wherein the floor includes a recessed area in a section that is located between the first and second catches, wherein when the tab is disposed in the first catch and the slider is in the closed position, a distal end of the slider is received in the recessed area of the floor.

17. The tamper evident cap of claim 12, wherein the second interlocking features further include a third catch that receives the tab to releasably retain the slider in an open position in which the luer tip is open and is accessible for filling of the syringe, the first catch being formed between the second and third catches.

18. The tamper evident cap of claim 17, wherein the first and third catches have the same shape that is different from a shape of the tab and each of the first, second and third catches comprises a recess formed in the ceiling that defines the slot and the tab is formed on and protrudes outwardly from an upper surface of the slider.

19. The tamper evident cap of claim 17, wherein a section of the ceiling between the first and third catches is planar and is parallel to a section of the floor that is opposite thereto, the ceiling including a beveled surface that declines in a direction away from the first catch and toward an opening of the slot.

20. The tamper evident cap of claim 1, wherein the tamper evident cap is placed on an empty syringe during a manufacturing process prior to delivery of a liquid to the syringe.

21. The tamper evident cap of claim 1, further including a tamper evident membrane that is disposed about a portion of the first section of the cap and about a portion of an empty syringe body for securing the tamper evident cap to the syringe during a process when the syringe is manufactured.

22. A tamper evident cap for placement on a syringe comprising:
a hollow bottom base section for receiving a luer tip of the syringe such that the base section surrounds the luer tip of the syringe;
an upper flange section that is integrally attached to the base section, the upper flange section having an opening formed therein that communicates with the base section and is located to provide an entrance into the luer tip when the cap is placed on the syringe, the upper flange section having a slot formed therein that is defined by a first surface and an opposing second surface; and
a slider having a locking tab formed as a part thereof, the slider being received within the slot and being movable in the slot between a first position where the slider is releasably retained in a closed position by reception of the locking tab in a first catch resulting in the luer tip being covered by the slider and not accessible through the flange section; a second position in which the luer tip is accessible through the opening of the upper flange section to permit filling of the syringe; and a third position where the slider is laterally locked in place by reception of the locking tab in a second catch resulting in the luer tip being covered by the slider and access to the luer tip being prevented, wherein movement of the slider in the slot subsequent to positioning the slider in the locked position causes the locking tab to break off of the slider, thereby indicating that the syringe has been tampered with subsequent to its filling.

23. The tamper evident cap of claim 22, further including a third catch that receives the locking tab to releasably retain the slider in the second open position and restrict lateral movement and prevents removal of the slide from the slot so long as the locking tab is integrally attached to the slider.

24. The tamper evident cap of claim 23, wherein the first catch comprises a first recess formed in the first surface, the second catch comprises a second recess formed in the first surface, and the third catch comprises a third recess formed in the first surface.

25. The tamper evident cap of claim 23, wherein the slider can be moved between the first position and the second position and from the first position to the third position and from the second position to the third position without the locking tab breaking off of the slider.

26. The tamper evident cap of claim 24, wherein a shape of the second catch is different from a shape of each of the first and third catches.

27. The tamper evident cap of claim 26, wherein a shape of the locking tab is complementary to the shape of the second catch but is different from the shapes of the first and third catches.

28. The tamper evident cap of claim 24, wherein the locking tab is defined by a convex surface having a radius of curvature that is substantially the same as a radius of curvature defining the second recess but is different from a radius of curvature defining each of the first and third recesses.

29. The tamper evident cap of claim 22, wherein the first and second catches are formed along the first surface, the first surface having a beveled section between the first and second catches that declines in a direction from the first catch towards the second catch to assist in the locking tab sliding within the slot towards and into engagement with the second catch.

30. The tamper evident cap of claim 22, wherein in the first position, the locking tab is only partially received in the first catch, while in the third position, substantially all of the locking tab is received in the second catch due to a height of ceiling in the area of the first catch, as measured from the floor, being greater than a height of the ceiling in the area of the second catch.

31. A tamper evident cap for placement on a syringe comprising:
a cap body for placement about a luer tip of the syringe, the cap body being a hollow member and including a slot formed in a side wall thereof, the slot being defined by an upper wall section and a lower wall section, the upper wall section having a through hole passing therethrough;
a cap top having a base section that is configured to be received within the slot, the base section having an opening formed therein, wherein when the cap top is fully inserted into the slot into a closed position, the cap top seals the cap body; and
a lock mechanism that is positionable within an open position to permit the cap top to be placed in the fill position as well as the closed position where the cap top seals the cap body but remains moveable relative to the cap body, and a locked position in which the cap top is securely attached to and seals the cap body.

32. The tamper evident cap of claim 31, wherein the lock mechanism includes a lock pin that is moveable between an open position where the lock pin is only inserted in the through hole and the cap top can be opened relative to the cap body and a locked position where the lock pin passes through and terminates within the opening formed in the base section so as to securely attach and lock the cap top in place relative to the cap body.

33. The tamper evident cap of claim 31, wherein the base section includes a pivot pin that is received into an opening in the lower wall section to permit the cap top to pivot relative to the cap body between the open fill position and the closed locked position.

34. The tamper evident cap of claim 32, wherein the lock pin has a head and a stem extending therefrom, the stem including a locking flange at a distal end thereof, the opening in the base section of the cap top including a groove that receives the locking flange so as to snap-fittingly lock the cap top to the cap body.

35. The tamper evident cap of claim 32, wherein the lock pin is formed of flexible plastic material.

36. The tamper evident cap of claim 32, wherein the opening formed in the base section includes an annular shaped groove at a closed end of the opening, the flange flexing into the groove for snap-fittingly locking the cap top to the cap body.

37. The tamper evident cap of claim 32, wherein the base section includes a projection formed on an upper surface thereof for mating with an inner edge that defines a through hole that passes through the hollow cap body to hold the cap top in the closed position prior to placing the lock pin in the locked position.

38. The tamper evident cap of claim 31, wherein the base section includes a projection on a lower surface thereof, the projection mating with an outer surface of a luer tip of the syringe so as to frictionally couple the cap body to the syringe.
